(19) United States
(12) Patent Application Publication
(10) Pub. No.: US 2004/0192734 A1
(43) Pub. Date: Sep. 30, 2004

(54) 4-(3-aminomethylphenyl)piperidin-1-yl]-[5-(2-fluorophenylethynyl)furan-2-yl]-methanone as an inhibitor of mast cell tryptase

(75) Inventors: Heinz W. Pauls, Oakville (CA); Suzanne Aldous, Gillette, NJ (US); Gregory H. Merriman, Phillipsburg, NJ (US); Robert A. Farr, Annandale, NJ (US); Adam W. Sledeski, Belle Mead, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 10/743,618

(22) Filed: Dec. 22, 2003

Related U.S. Application Data
(60) Provisional application No. 60/436,534, filed on Dec. 26, 2002.

(51) Int. Cl. C 07D 4 5/02; A61K 31/452

(52) U.S. Cl. 514/326; 546/207

(57) ABSTRACT

The present invention extends to the compound of formula (I):

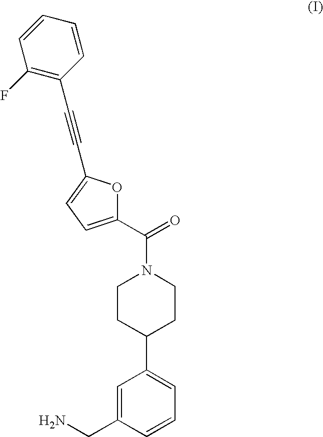

or a prodrug, pharmaceutically acceptable salt, or solvate of said compound; to a pharmaceutical composition comprising a pharmaceutically effective amount of the compound of formula (I), and a pharmaceutically acceptable carrier; the use of a compound of formula (I) as an inhibitor of tryptase comprising introducing the compound into a composition comprising tryptase.

### FIELD OF THE INVENTION

This invention is directed to a substituted arylmethylamine compound, its preparation, a pharmaceutical composition comprising the compound, its use, and intermediates thereof.

### BACKGROUND OF THE INVENTION

Mast cell mediated inflammatory conditions, in particular asthma, are a growing public health concern. Asthma is frequently characterized by progressive development of hyper-responsiveness of the trachea and bronchi to both immunospecific allergens and generalized chemical or physical stimuli, which lead to the onset of chronic inflammation. Leukocytes containing IgE receptors, notably mast cells and basophils, are present in the epithelium and underlying smooth muscle tissues of bronchi. These leukocytes initially become activated by the binding of specific inhaled antigens to the IgE receptors and then release a number of chemical mediators. For example, degranulation of mast cells leads to the release of proteoglycans, peroxidase, arylsulfatase B, chymase, and tryptase, which results in bronchiole constriction.

Tryptase is stored in the mast cell secretory granules and is the major secretory protease of human mast cells. Tryptase has been implicated in a variety of biological processes, including degradation of vasodilating and bronchorelaxing neuropeptides (Caughey, et al., J. Pharmacol. Exp. Ther., 1988, 244, pages 133-137; Franconi, et al., J. Pharmacol. Exp. Ther., 1988, 248, pages 947-951; and Tam, et al., Am. J. Respir. Cell Mol. Biol., 1990, 3, pages 27-32) and modulation of bronchial responsiveness to histamine (Sekizawa, et al., J. Clin. Invest., 1989, 83, pages 175-179).

As a result, tryptase inhibitors may be useful as anti-inflammatory agents (K Rice, P. A. Sprengler, Current Opinion in Drug Discovery and Development, 1999, 2(5), pages 463-474) particularly in the treatment of chronic asthma (M. Q. Zhang, H. Timmerman, Mediators Inflamm., 1997, 112, pages 311-317), and may also be useful in treating or preventing allergic rhinitis (S. J. Wilson et al, Clin. Exp. Allergy, 1998, 28, pages 220-227), inflammatory bowel disease (S. C. Bischoff et al, Histopathology, 1996, 28, pages 1-13), psoriasis (A. Naukkarinen et al, Arch. Dermatol. Res., 1993, 285, pages 341-346), conjunctivitis (A. A. Irani et al, J. Allergy Clin. Immunol., 1990, 86, pages 34-40), atopic dermatitis (A. Jarvikallio et al, Br. J. Dermatol., 1997, 136, pages 871-877), rheumatoid arthritis (L. C Tetlow et al, Ann. Rheum. Dis., 1998, 54, pages 549-555), osteoarthritis (M. G. Buckley et al, J. Pathol., 1998, 186, pages 67-74), gouty arthritis, rheumatoid spondylitis, and diseases ofjoint cartilage destruction.

In addition, tryptase has been shown to be a potent mitogen for fibroblasts, suggesting its involvement in the pulmonary fibrosis in asthma and interstitial lung diseases (Ruoss et al., J. Clin. Invest., 1991, 88, pages 493-499).

Therefore, tryptase inhibitors may be useful in treating or preventing fibrotic conditions (J. A. Cairns and A. F. Walls, J. Clin. Invest., 1997, 99, pages 1313-1321) for example, fibrosis, sceleroderma, pulmonary fibrosis, liver cirrhosis, myocardial fibrosis, neurofibromas and hypertrophic scars.

Additionally, tryptase inhibitors may be useful in treating or preventing myocardial infarction, stroke, angina and other consequences of atherosclerotic plaque rupture (M. Jeziorska et al, J. Pathol., 1997, 182, pages 115-122).

Tryptase has also been discoveredto activate prostromelysin that in turn activates collagenase, thereby initiating the destruction of cartilage andperiodontal connective. tissue, respectively.

Therefore, tryptase inhibitors could be useful in the treatment or prevention of arthritis, periodontal disease, diabetic retinopathy, and tumor growth (W. J. Beil et al, Exp. Hematol., (1998) 26, pages 158-169). Also, tryptase inhibitors may be useful in the treatment of anaphylaxis (L. B. Schwarz et al, J. Clin. Invest., 1995, 96, pages 2702-2710), multiple sclerosis (M. Steinhoffet al, Nat. Med. (N.Y.), 2000, 6(2), pages 151-158), peptic ulcers and syncytial viral infections.

Substituted arylmethylamines, represented as the compounds of formula (A), their preparation, pharmaceutical compositions containing these compounds, and their pharmaceutical use in the treatment of disease states capable of being modulated by the inhibition of tryptase are reported in pending U.S. application Ser. No. 09/843,126. Encompassed within the generic disclosure of the compounds of formula (A) of U.S. application Ser. No. 09/843,126, is the compound of the present invention, formula (I). However, the compound of formula (I) is not specifically disclosed in U.S. Application Ser. No. 09/843,126.

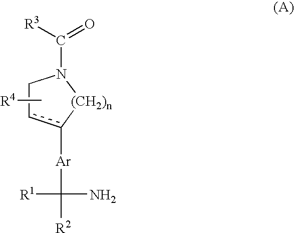

Accordingly, what is needed is a novel and useful compound having particularly valuable pharmaceutical properties, in its ability to inhibit tryptase. Such a compound should readily have a utility in treating a patient suffering from conditions that can be ameliorated by the administration of an inhibitor of tryptase, e.g., mast cell mediated inflammatory conditions, inflammation, and diseases or disorders related to the degradation of vasodilating and bronchorelaxing neuropeptides.

### SUMMARY OF THE INVENTION

The present inventionextends to the compound of fonriula (I):

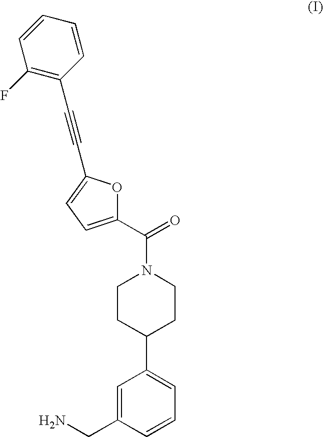

or a prodrug, pharmaceutically acceptable salt, or solvate of said compound.

Furthermore, the present invention is directed to a pharmaceutical composition comprising a pharmaceutically effective amount of the compound of formula (I), and a pharmaceutically acceptable carrier.

Furthermore, the present invention is directed to the use of a compound of formula (I) as an inhibitor of tryptase, comprising introducing the compound into a composition comprising tryptase.

### DETAILED DESCRIPTION

### Definitions

As used above, and throughout the instant specification and appending claims, the following terms, unless otherwise indicated, shall be understood to have the following meanings:

As used herein, the term “compound of the present invention”, and equivalent expressions, are meant to embrace the compound of formula (I), as hereinbefore described, which expression includes the prodrug, the pharmaceutically acceptable salt and the solvate, e.g., hydrate. Similarly, reference to intermediates, whether or not they themselves are claimed, is meant to embrace the salts, and solvates, where the context so permits. For the sake of clarity, particular instances when the context so permits are sometimes indicated in the text, but these instances are purely illustrative and they are not intended to exclude other instances when the context so permits.

As used herein, the term “treatment” or “treating” includes prophylactic therapy as well as treatment of an established condition.

“Patient” means a human or other mammal.

“Effective amount” is meant to describe an amount of a compound effective in producing the desired therapeutic effect.

“Prodrug” means a compound which is suitable for administration to a patient without undue toxicity, irritation, allergic response, and the like; and is convertible in vivo by metabolic means (e.g. by hydrolysis) to the compound of the present invention: A thorough discussion of prodrugs is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987, both of which are incorporated herein by reference.

### Particular or Preferred Embodiments

In addition, the present invention is directed to the use of the compound of formula (I) for treating a patient suffering from a physiological condition that can be ameliorated by administering to the patient a therapeutically effective amount of the compound of formula (I). Particular embodiments of physiological conditions that can be treated with the compound of the present invention include, but certainly are not limited to inflammatory diseases, e.g., joint inflammation, arthritis, rheumatoid arthritis, rheumatoid spondylitis, gouty arthritis, traumatic arthritis, rubella arthritis, psoriatic arthritis, and other chronic inflammatory joint diseases. Other embodiments of physiological conditions that can be treated by the present invention include physiological conditions such as joint cartilage destruction, ocular conjunctivitis, vernal conjunctivitis, inflammatory bowel disease, asthma, allergic rhinitis, interstitial lung diseases, fibrosis, sceleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina and other consequences of atherosclerotic plaque rupture, as well as periodontal disease, diabetic retinopathy, tumor growth, anaphylaxis, multiple sclerosis, peptic ulcers, and syncytial viral infections.

In a particular embodiment, the present invention is directed to the use of a compound of formula (I) for treating a patient suffering from asthma, comprising administering to the patient a physiologically effective amount of the compound.

Another special embodiment of the therapeutic method of the present invention is treatment of allergic rhinitis.

In another particular embodiment, the present invention is directed to the use of a compound of formula (I) for treating a patient suffering from joint inflammation, comprising administering to the patient a physiologically effective amount of the compound.

In another particular embodiment, the present invention is directed to the use of a compound of formula (I) for treating a patient suffering from inflammatory bowel disease, comprising administering to the patient a physiologically effective amount of the compound.

In addition, the present invention extends to a pharmaceutical composition comprising the compound of formula (I), a second compound selected from the group consisting of a beta andrenergic agonist, an anticholinergic, an anti-inflammatory corticosteroid; and an anti-inflammatory agent, and a pharmaceutically acceptable carrierthereof. In such a coinpositidn the compound of formula (I) and the second compound are present in amounts such that provide a therapeutically efficacious-activity, i.e., additive or synergistic effect. Particular inflammatory diseases or disorders that can be treated with such a pharmaceutical composition include, but is not limited to, asthma.

Moreover, the present invention is directed to a method for treating a patient suffering from an inflammatory disorder, comprising administering to the patient the compound of formula (I) and a second compound selected from the group consisting of a beta andrenergic agonist, an anticholinergic, an anti-inflammatory corticosteroid, and an anti-inflammatory agent. In such a method, the compound of formula (I) and the second compound are present in amounts such that provide a therapeutically efficacious activity, i.e., additive or synergistic effect. In such a method of the present invention, the compound of the present invention can be administered to the patient before a second compound, a second compound can be administered to the patient before a compound of the present invention, or a compound of the present invention and a second compound can be administered concurrently. Particular examples of andrenergic agonists, anticholinergics, anti-inflammatory corticosteroids, and anti-inflammatory agents having application according to the method are described infra.

### Pharmaceutical Compositions

As explained above, the compound of the present invention exhibits useful pharmacological activity and accordingly may be incorporated into a pharmaceutical composition and used in the treatment of patients suffering from certain medical disorders. The present invention thus provides, according to a further aspect, pharmaceutical compositions comprising the compound of the invention, and a pharmaceutically acceptable carrier thereof. As used herein, the term “pharmaceutically acceptable” preferably means approved by a regulatory agency of a government, in particular the Federal government or a state government, or listed in the U.S. Pharmacopeia or another generally recognized pharmacopeia for use in animals, and more particularly in humans. Suitable pharmaceutical carriers are described in “Remington's Pharmaceutical Sciences” by E. W. Martin.

Pharmaceutical compositions according to the present invention can be prepared according to the customary methods, using one or more pharmaceutically acceptable adjuvants or excipients. The adjuvants comprise, inter alia, diluents, sterile aqueous media and the various non-toxic organic solvents. The compositions may be presented in the form of tablets, pills, granules, powders, aqueous solutions or suspensions, injectable solutions, elixirs or syrups, and can contain one or more agents chosen from the group comprising sweeteners, flavorings, colorings, or stabilizers in order to obtain pharmaceutically acceptable preparations. The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents. such as starch, alginic acids and certain complex, silicates combined with lubricants such as magnesium stearate, sodium.lauryl sulfate and talc may be used for preparingtablets. To prepare: a capsule, it is advantageous to. use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used. Such pharmaceutically acceptable carriers can also be sterile water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include mannitol, human serum albumin (HSA), starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol and the like. These compositions can take the form of solutions, suspensions, tablets, pills, capsules, powders, sustained-release formulations and the like.

Naturally, a pharmaceutical composition of the present invention will contain a therapeutically effective amount of the active compound together with a suitable amount of carrier so as to provide the form for proper administration to the patient. While intravenous injection is a very effective form of administration, other modes can be employed, such as by injection, or by oral, nasal or parenteral administration, which are discussed infra.

### Methods of Treatment

The compound of formula (I) possesses tyyptase inhibition activity according to tests described in the literature and described hereinafter, and which test results are believed to correlate to pharmacological activity in humans and other mammals. Thus, in a further embodiment, the present invention is directed to the use of formula (I) or a composition comprising it for treating a patient suffering from, or subject to, a condition that can be ameliorated by the administration of an inhibitor of tryptase. For example, the compound of formula (I) is useful for treating an inflammatory disease, for example, joint inflammation, including arthritis, rheumatoid arthritis and other arthritic condition such as rheumatoid spondylitis, gouty arthritis, traumatic arthritis, rubella arthritis, psoriatic arthritis, osteoarthritis or other chronic inflammatory joint disease, or diseases of joint cartilage destruction, ocular conjunctivitis, vernal conjunctivitis, inflammatory bowel disease, asthma, allergic rhinitis, interstitial lung diseases, fibrosis, sceleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina or other consequences of atherosclerotic plaque rupture, as well as periodontal disease, diabetic retinopathy, tumor growth, anaphylaxis, multiple sclerosis, peptic ulcers, or a syncytial viral infection.

According to a further feature of the invention there is provided a method for the treatment, of a human or animal patient suffering from, or subject to, conditions which can be ameliorated by the administration of an inhibitor. of tryptase, for example conditions as hereinbefore described, which comprises the administration to the patient of an effective amount of compound of the invention or a composition containing a compound of the invention.

### Combination Therapy

As explained above, other pharmaceutically active agents can be employed in combination with the compound of formula (I) depending upon the disease being treated. For example, in the treatment of asthma, beta-adrenergic agonists such as albuterol, terbutaline, formoterol, fenoterol or prenaline can be included, as can anticholinergics such as ipratropium bromide, anti-inflammatory corticosteroids such as beclomethasone dipropionate, triamcinolone acetonide, flunisolide or dexamethasone, and anti-inflammatory agents such as sodium cromoglycate and nedocromil sodium. Thus, the present invention extends to a pharmaceutical composition comprising the compound of formula (I) and a second compound selected from the group consisting of a beta andrenergic agonist, an anticholinergic, an anti-inflammatory corticosteroid, and an anti-inflammatory agent; and a pharmaceutically acceptable carrier thereof. Particular pharmaceutical carriers having applications in this pharmaceutical composition are described herein.

Furthermore, the present invention extends to a method for treating a patient suffering from asthma, comprising administering the patient the compound of the present invention, and a second compound selected from the group consisting of a beta andrenergic agonist, an anticholinergic, an anti-inflammatory corticosteroid, and an anti-inflarnmatory agent. In such a combination method, the compound of the present invention can be administered prior to the administration of the second compound, the compound of the present invention can be administered after administration of the second compound, or the compound of the present invention and the second compound can be administered concurrently.

### Modes of Delivery

According to the invention, the compound of formula (I), or a pharmaceutical composition comprising the compound, may be introduced parenterally, transmucosally, e.g., orally, nasally, pulmonarily, or rectally, or transdermally to a patient.

### Oral Delivery

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers,(e.g., U.S. Pat. No. 5,013,556). A description of possible solid dosage forms. for a therapeutic is given by Marshall, K. In: Modern Pharmaceutics Edited by G. S. Banker and C. T. Rhodes Chapter 10, 1979, herein incorporated by reference. In general, the formulation will include a compound of the present invention, and inert ingredients which allow for protection against the stomach environment, and release of the biologically active material, i.e., a compound of the present invention, in the intestine.

Also specifically contemplated are oral dosage forms of the compound of the present invention. Such a compound may be chemically modified so that oral delivery is more efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the compound of the present invention, and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the compound of the present invention, the location of release may be the stomach, the small intestine (the duodenum, the jejunum, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the compound of the present invention, or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings that make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation.of the material for capsule administration could also be as a powder, lightly compressed plugs or even as tablets. The therapeutic could be prepared by compression.

Colorants and flavoring agents may all be included. For example, the compound of the present invention may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include, but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added

as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of a compound of the present invention either alone or as a mixture in different ratios.

Additives which potentially enhance uptake of the compound of the present invention are, for instance, the fatty acids oleic acid, linoleic acid and linolenic acid. Controlled release oral formulation may be desirable. The drug could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan-coater or in a fluidized bed or by compression coating.

### Pulmonary Delivery

Also contemplated herein is pulmonary delivery of the compound of the present invention, either alone, or in a pharmaceutical composition. The compound is delivered to the lungs of a mammal while inhaling and traverses across the lung epithelial lining to the blood stream. Other reports of this include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass., to name only a few. All such devices require the use of formulations suitable for the dispensing of the compound of the present invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. A chemically modified compound of the present invention may also be prepared in different formulations depending on the type of chemical modification or the type of device employed.

Formulations suitable for use with a nebulizer, either jet or ultrasonic, will typically comprise the compound of the present invention dissolved in water at a concentration of about 0.1 to 25 mg of compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the compound of the invention suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the compound of the invention, and may also include a bulking agent, such as lactose, sorbitol, sucrose, or mannitol in amounts which facilitate dispersal of the powder from the. device, e.g., 50 to 90% by weight of the formulation. The compound of the present invention should most advantageously be prepared in particulate form with an average particle size of less than 10 mm (or microns), most preferably 0.5 to 5 mm, for most effective delivery to the distal lung.

### Nasal Delivery

Nasal delivery of the compound of the present invention is also contemplated. Nasal delivery allows the passage of the compound to the blood stream directly after administering the therapeutic product to the nose, without the necessity for deposition of the product in the lung. Formulations for nasal delivery include those with dextran or cyclodextran.

### Transdermal Delivery

Various and numerous methods are known in the art for transdermal administration of a drug, e.g., via a transdermal patch, have applications in the present invention. Transdermal patches are described in for example, U.S. Pat. No. 5,407,713, issued Apr. 18, 1995 to Rolando et al.; U.S. Pat. No. 5,352,456, issued Oct. 4, 1004 to Fallon et al.; U.S. Pat. No. 5,332,213 issued Aug. 9, 1994 to D'Angelo et al.; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al.; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al.; and U.S. Pat. No. 4,921,475, issued May 1, 1990 to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

### Topical Administration

For topical administration, gels (water or alcohol based), creams or ointments containing compounds of the invention may be used. Compounds of the invention may also be incorporated in a gel or matrix base for application in a patch, which would allow a controlled release of compound through the transdermal barrier.

### Rectal Administration

Solid compositions for rectal administration include suppositories formulated in accordance with known methods and containing the compound of the invention.

### Dosages

The percentage of active ingredient in the composition of the invention may be varied, it being necessary that it should constitute a proportion such that a suitable dosage shall be obtained. Obviously, several unit dosage forms may be administered at about the same time. The dose employed will be determined by the physician, and depends upon the desired therapeutic. effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 1, mg/kg body weight per day by intravenous administration. In each particular case, the doses will be determined in accordance with the factors distinctive to the subject to be treated, such as age, weight, general state of health and other characteristics which can influence the efficacy of the medicinal product.

Furthermore, the compound according to the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. Of course, for some patients, it will be necessary to prescribe not more than one or two doses per day.

Naturally, a patient in whom administration of the compound of the present invention is an effective therapeutic regimen is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

### Preparatory Details

The compound of formula (I) may be prepared by the application or adaptation of known methods, by which is meant methods used heretofore or described in the literature, for example those described by R. C. Larock in Comprehensive Organic Transformations, VCH publishers, 1989, or as described herein.

In the reactions described hereinafter it may be necessary to protect reactive functional groups, for example, amino groups, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991. In particular, the compound of formula (I) may be prepared as shown through Schemes 1, 2, and 3.

For example, the compound of the present invention is an achiral compound whose preparation is comprised of a convergent synthesis. A six-step sequence culminates in the preparation of amine 8, which is coupled, to acid chloride 13 (prepared through a three step sequence), to yield the compound of formula (I) in a two-step sequence. The preparation of 8, 13 and the final compound (I) of the present invention will be discussed in turn.

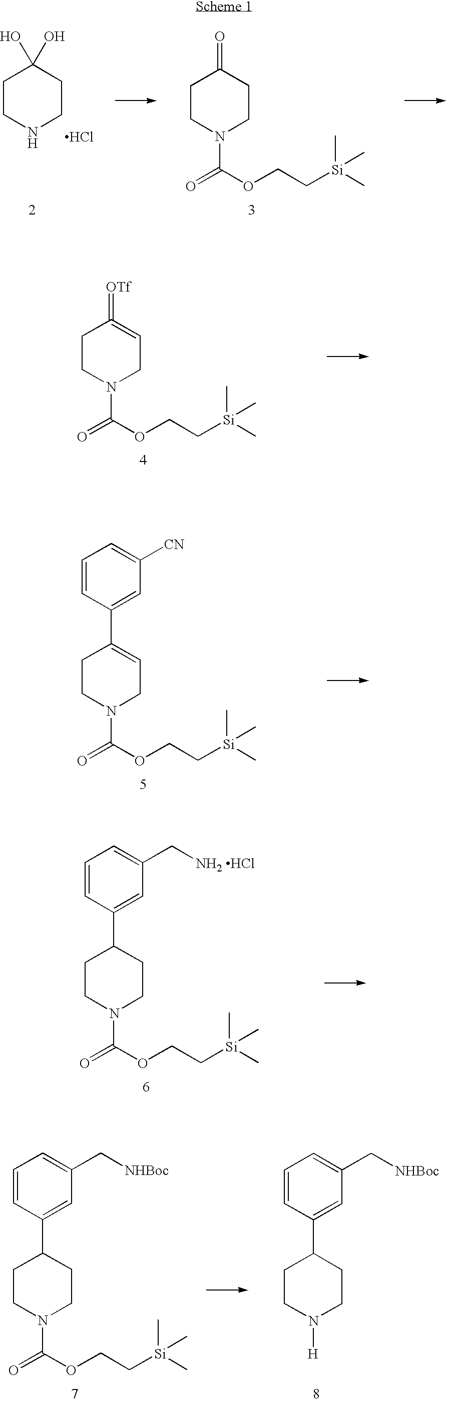

For example, the amine compound of formula (8) may be prepared as follows: The first step, protection of the 4-piperidone (2) as the N-Teoc (2-(trimethylsilyl)ethoxycarbonyl) derivative 3, utilizes 2-(trimethylsilyl)ethyl p-nitrophenyl carbonate. Alternatively, this reagent may be replaced by the in situ generation of the carbamate formed from 1,1′-carbonyldulmidazole and 2-(trimethylsilyl)ethanol. The formation of this reagent is mildly exothermic, with a rise of ˜10° C. which is easily controlled by the rate of addition of the 2-(trimethylsilyl)ethanol. Kugelrohr distillation of ketone 3 at 156-160° C. under high vacuum gives 3 as a colorless liquid. Enolization of ketone 3 at −78° C. with Li(TMS)₂ in THF, followed by quenching the enolate with N-phenyl-bis(trifluoromethanesulfonimide) gives the vinyl triflate 4. Triflate 4 is not purified, but used immediately in a Suzuki coupling with 3-cyanophenylboronic acid in refluxing CH₃CN/2 M Na₂CO₃ and tetrakistriphenylphosphine Pd (0) to give nitrile 5. This step requires chromatography through a short plug of silica gel to remove the Pd catalyst and very polar materials. This minimal chromatographic purification is sufficient, since minor impurities are removed in a later crystallization of 7. One impurity formed is the homo-coupled product, 3,3′-dicyanobiphenyl. This highly crystalline compound precipitates from later fractions upon removal of the solvent and can be partially removed by addition of cyclohexane to the mixture followed by filtration. Hydrogenation of both the nitrile and the double bond using 10% Pd/C in the presence of 1+ equivalents of HCl in EtOH gives the amine hydrochloride. Removal of the EtOH solvent gives a semi-solid, which is washed free of partially reduced nitrile with ether/EtOAc to give 6. Protection of the primary amine as the N-Boc derivative gives carbamate 7 as a crystalline solid. Carbamate 7 is easily separated from impurities by trituration with pentane; it can alternatively be recrystallized from cyclohexane/hexane. Removal of the N-Teoc with 1 M (n-Bu)₄NF/[HF at 50° C. gives, after trituration with ether/pentane, amine 8 as a colorless solid in 94% yield.

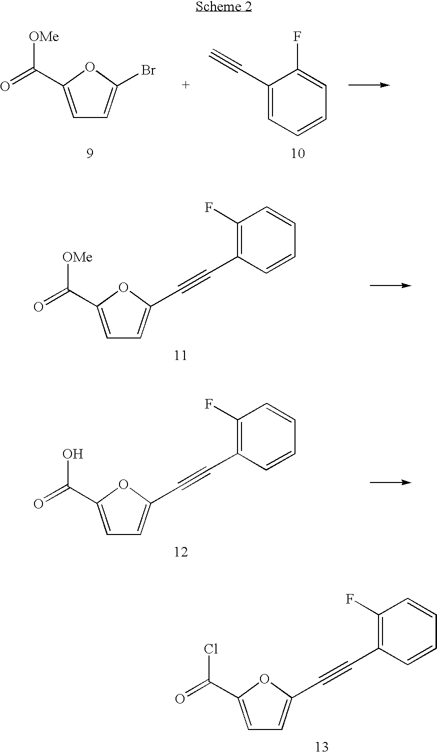

The acid chlorides compound of formula (13) may be prepared as follows: Ethynyl-2-fluorobenzene 10 is coupled to methyl 4-bromo-2-furanoate via a high yielding palladium mediated process. Standard work-up and chromatography yields 11, which is saponified under standard conditions. The crude product is recrystallized (EtOH/water) to give acid 12. Conversion to the acid chloride 13 is accomplished with oxalyl chloride/catalytic DMF in CH₂Cl₂. Removal of the solvent gives crude 13 as an olive solid in quantitative yield.

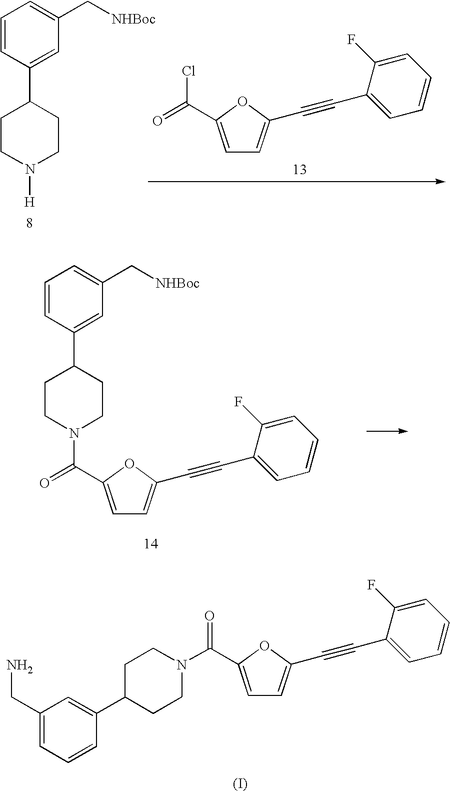

The final compound of formula (I) as the methanesulfonate salt may be prepared as follows: Acylation of amine 8 with acid chloride 13 gives the N-Boc acetylene 14. The crude product is purified by flash chromatography followed by trituration with ice-cold ether/pentane. Alternatively, compound 14 can be recrystallized from 10:1 cyclohexane/EtOAc. Evaporation of the solvent from a solution of 14 and 1 equivalent of methanesulfonic acid in i-PrOH while heating to 60° C. under vacuum removed the N-Boc to give the compound of formula (I) as the methanesulfonate salt. The compound of formula (I) as the methanesulfonate salt crystallizes upon trituration with acetone and was recrystallized from i-PrOH/CH₃CN. The final deprotection can also be effected by TFA/CH₂Cl₂ to yield a brown gum.

The compound of the present invention is basic, and such compound is useful in the form of the free base or in the form of a pharmaceutically acceptable acid addition salt thereof.

Acid addition salts may be a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Although pharmaceutically acceptable salts of said basic compound is preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. Pharmaceutically acceptable salts within the scope of the invention include those derived from mineral acids and organic acids, and include hydrohalides, e.g. hydrochlorides and hydrobromides, sulfates, phosphates, nitrates, sulfamates, acetates, citrates, lactates, tartrates, malonates, oxalates, salicylates, propionates, succinates, fumarates, maleates, methylene-bis-b-hydroxynaphthoates, benzoates, tosylates, gentisates, isethionates, di-p-toluoyltartrates, methanesulfonates, ethanesulfonates, benzenesulfonates, p-toluenesulfonates, cyclohexylsulfamates and quinates.

As well as being useful in itself as an active compound, salts of the compound of the invention are usefuil for the purposes of purification of the compound, for example by exploitation of the solubility differences between the salts and the parent compound, side products and/or starting materials by techniques well known to those skilled in the art.

According to a further feature of the invention, the acid addition salt of the compound of this invention may be prepared by reaction of the free base with the appropriate acid, by the application or adaptation of known methods. For example, the acid addition salts of the compound of this invention may be prepared either by dissolving the free base in water or aqueous alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compound of this invention can be regenerated from the salts by the application or adaptation of known methods. For example, the parent compound of the invention can be regenerated from their acid addition salts by treatment with an alkali, e.g. aqueous sodium bicarbonate solution or aqueous ammonia solution.

The starting materials and intermediates may be prepared by the application or adaptation of known methods, for example methods as described in the Reference Examples or their obvious chemical equivalents.

The present invention is also directed to some intermediates in the above schemes and, as such, the processes described herein for their preparation constitute further features of the present invention.

### EXAMPLES

The present invention may be better understood by reference to the following non-limiting Examples, which are provided as exemplary of the invention. The following examples are presented in order to more fully illustrate particular embodiments of the invention. They should in no way be construed, however, as limiting the broad scope of the invention.

In the nuclear magnetic resonance spectra (NMR), reported infra, the chemical shifts are expressed in ppm relative to tetramethylsilane. Abbreviations have the following significances: br=broad, dd=double doublet, s=singlet; m=multiplet.

### Example 1

### [4-(3 -Aminomethylphenyl)-piperidin-1 -yl]-[5 -(2-fluorophenylethynyl)-furan-2-yl]-methanone trifluoroacetate [which may also be named as 3-(1-[5-(2-fluorophenylethynyl)-furan-2-carbonyl]-piperidine-4-yl)-benzylamine trifluoroacetate]

A. 4-Oxo-piperidine-carboxylic acid 2-trimethylsilanyl-ethyl ester

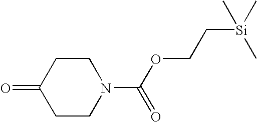

A solution of 13.55 g (88.21 mmol) of 4-piperidone monohydrate hydrochloride (25 g, 88.22 mmol), 2-trimethylsilylethyl p-nitrophenylcarbonate (50 niL, 359.7 mmol), triethylamine (50 mL, 0.345 mol) and DMAP (10.78 g, 88.24 mmol) in 300 mL of acetonitrile is warmed under reflux for 2 hours and then allowed to cool to room temperature. The mixture is diluted with 300 mL of dichloromethane and washed 3×100 mL of 1 M HCl and 4×100 mL of 1M NaOH until all of the yellow color is removed from the organic phase. The organic phase is then washed with brine and dried over MgSO₄. The organic phase is concentrated in vacuo to afford 19.35 g (90%) of the title, compound as a colorless oil. ¹H NMR (CDCl₃) δ 4.22 (m, 2H), 3.75 (t, 4H, J=6.2 Hz), 2.44 (t,4H, J=6.2 Hz), 1.02 (m, 2H), 0.04 (s, 9H).

B. 4-(3-Cyanophenyl)-3,6-dihydro-2H-pyridine-1-carboxylic acid 2-trimethylsilanyl-ethyl ester

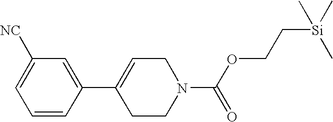

To a flask containing 50 mL of tetrahydrofuran at −70° C. is added 60 mL (60 mmol) of 1M lithium hexamethyldisilazide dropwise. A solution of 13.3 g (55 mmol) of 4-Oxo-piperidine-1-carboxylic acid 2-trimethylsilanyl-ethyl ester is then added via dropping funnel over 20 minutes keeping the internal temperature between −65° C. and −70° C. The solution is stirred at −70° C. for 45 minutes then a solution of 19.65 g (55 mmol) of phenyltrifluoromethane sulfonamide in 75 mL of tetrahydrofuran is added dropwise over 20 minutes. The solution was allowed to warm to 0° C. and stirred for 3 hours. The reaction is then concentrated in vacuo and the residue, 4-trifluoromethanesulfonyloxy-3,6-dihydro-2H-pyridine-1-carboxylic acid 2-trimethyl-silanyl-ethyl ester, is used without further purification.

To a solution of 20.65 g (55 mmol) of 4-trifluoromethanesulfonyloxy-3,6-dihydro-2H-pyridine-1-carboxylic acid 2-trimethyl-silanyl-ethyl ester in 300 mL of acetonitrile is added 8.9 g (60.6 mmol) of 3-cyanophenylboronic acid followed by 82.5 mL (165 mmol) of 2M sodium carbonate, 6.98 g (165 mmol) of lithium chloride and 3.18 g (2.8 mmol) of tetrakistriphenylphosphine Palladium (0). The mixture is warmed under reflux for 90 minutes then allowed to cool to room temperature and filtered. The filtrate is concentrated and diluted with 300 mL of 2 M Na₂CO₃ then extracted 3×dichloromethane. The organic phase is washed with brine then separated and dried (MgSO₄). The organic phase is concentrated in vacuo and the crude residue is flash chromatographed over SiO₂ (eluted with Heptane:EtOAc:DCM=5:1:1) to give 10.46 g (58%) of the title compound as a yellow oil. ¹H NMR (CDCl₃) δ 7.65-7.52 (m, 3H), 7.44 (t, 1H, J=7.7 Hz), 6.11 (bs, 1H), 4.23 (m, 2H), 4.15 (m, 2H), 3.70 (t, 2H, J=5.6 Hz), 2.52 (m, 2H), 1.04 (m, 2H), 0.06 (s, 9H).

C. 4-(3-Aminomethyl-phenyl)-piperidine-1-carboxylic acid 2-trimethylsilanyl-ethyl ester

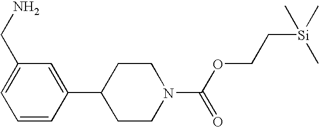

To a slurry of 5 g of 10% Pd/C (wet) in 250 mL of ethanol is added 2.9 mL (34.8 mmol) of concentrated HCl and 10.4 g of 4-(3-Cyanophenyl)-3,6-dihydro-2H-pyridine-1-carboxylic acid 2-trimethylsilanyl-ethyl ester. The mixture is hydrogentated at 50 psi for 4 hours. The mixture is then filtered over a cake of celite and the cake is washed with excess ethanol. The filtrate is then concentrated in vacuo and the residue is triturated with Et₂O/pentane, then filtered to give 7.1 g of the title compound as a white solid. ¹H NMR (CD₃OD) δ 7.41-7.27 (m, 4H), 4.26 (dm, 2H, J=13.5 Hz), 4.20 (m, 2H), 4.09 (s, 2H), 2.92 (bm, 2H), 2.79 (tt, 1H, J=12.1, 3.6 Hz), 1.84 (dm, 2H, J=12.9 Hz), 1.62 (qd, 2H, J=12.6, 4.1 Hz), 1.02 (m, 2H), 0.06 (s, 9H); MS (APCI, MeOH/H₂O) m/z 336, 335 (M⁺+1, 100), 191.

D. 4-[3-(tert-Butoxycarbonylamino-methyl)-phenyl]-piperidine-1-carboxylic acid 2-trimethylsilanyl-ethyl ester

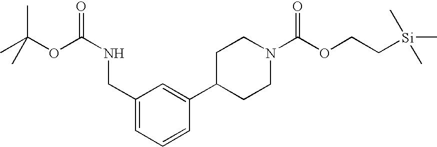

To a solution of 4-(3-Aminomethyl-phenyl)-piperidine-1-carboxylic acid 2-trimethylsilanyl-ethyl ester (11.1 g, 29.93 mmol) in 150 mL of dichloromethane and 50 mL of saturated NaHCO₃ is added Boc-anhydride (6.54 g, 29.96 mmoL). The mixture is stirred overnight at room temperature. The organic phase is then separated and washed with water and brine. The organic phase is then separated, dried (MgSO₄) and concentrated in vacuo to give 13.41 g (100%) of the title compound as an oil. ¹H NMR (CDCl₃) δ 7.26 (m, 1H), 7.10 (m, 3H), 4.85 (bs, 1H), 4.29 (d, 4H, J=5.8 Hz), 4.19 (m, 2H), 2.83 (t, 2H, J=12.5 Hz), 2.64 (tt, 1H, J=12.0, 3.6 Hz), 1.81 (m, 2H), 1.60 (m, 2H), 1.45 (s, 9H), 1.01 (t, 2H, J=8.4 Hz), 0.04 (s, 9H).

E. (3-Piperidin-4-yl-benzyl)-carbamic acid tert-butyl ester

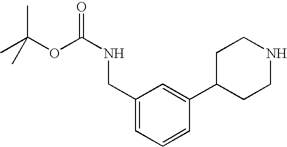

To a solution of 13.41 g (30.9 mmol) of 4-(3-tert-butoxycarbonylaminomethylphenyl)-piperidine-1-carboxylic acid 2-trimethylsilanyl-ethyl ester in 200 mL of tetrahydrofuran is added 34 mL (34 mmol) of tetrabutyl ammonium fluoride (1M). The mixture is warmed to 50° C. for 2 hours then allowed to cool to room temperature and stand overnight. To complete the reaction the mixture is heated for an additional 3 h at 50° C. The mixture is then concentrated in vacuo, diluted with 1M HCl and extracted with Et₂O. The aqueous phase is made basic with 1N NaOH and extracted 3× with EtOAc. The organic phases are combined, washed with brine, separated and dried (MgSO₄). The organic phase is filtered and concentrated in vacuo to afford 8.3 g (93%) of the title compound as a yellow oil which is used without further purification. ¹H NMR (300 MHz, CDCl₃) δ 7.25 (m, 1H), 7.07-7.13 (m, 3H), 4.85 (bs, 1H), 4.29 (d, 2H, J=5.1 Hz), 3.17 (dm, 2H, J=12.0 Hz), 2.72 (td, 2H, J=12.0, 2.4 Hz), 2.60 (tt, 1H, J=12.0, 3.6 Hz), 1.81 (m, 2H), 1.55-1.70 (m, 3H), 1.46 (s, 9H).

F. 5-(2-Fluoro-phenylethynyl)-furan-2-carboxylic acid ethyl ester

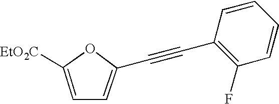

A solution of 3.34 g (15.25 mmol) of 5-bromo-2-furoic acid ethyl ester, 2.38 mg (19.8 mmol) of 1-ethynyl-2-fluorobenzene, 144 mg (0.76 mmol) of copper (I) iodide and 533 mg (0.76 mmol) of bistriphenylphosphine palladium dichloride in 50 mL of triethylamine is warmed to 60° C. and stirred for 3 hours. The mixture is diluted with EtOAc and filtered through celite. The mixture is concentrated in vacuo and the residue is flash chromatographed over 90 g of SiO₂ (eluted with Hept:EtOAc=9:1 followed by Hept:EtOAc=4:1) to give 3.3 g (84%) of the title compound as an off white solid. ¹H NMR (300 MHz, DMSO-d₆) δ 7.66 (m, 1H), 7.53 (m, 1H), 7.38 (d, 1H, J=3.7 Hz), 7.35 (m, 1H), 7.28 (t, 1H, J=7.6 Hz), 7.11 (d, 1H, J=3.7 Hz), 3.83 (s, 3H).

G. 5-(2-Fluoro-phenylethynyl)-furan-2-carboxylic acid

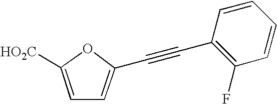

To a solution of 4.5 g (17.4 mmol) of 5-(2-Fluoro-phenylethynyl)-furan-2-carboxylic acid ethyl ester in 100 mL of MeOH:THF=1:4 is added 10 mL of 10% aqueous NaOH. The mixture is stirred for 5 hours at room temperature, then diluted with 500 mL of EtOAc and washed with 200 mL of water. The aqueous phase is acidified to pH=1 using 2 N HCl, then extracted with 3×200 mL of EtOAc. The organic phases are combined, dried (MgSO₄) and concentrated in vacuo to afford 3.62 g (90%) of the title compound as a white solid. ¹H NMR (300 MHz, DMSO-d₆) δ 13.4 (bs, 1H), 7.69 (m, 1H), 7.55 (m, 1H), 7.38 (t, 1H, J=9.0 Hz), 7.32 (d, 1H, J=3.8 Hz), 7.31 (t, 1H, J=7.5 Hz), 7.11 (d, 1H, J=3.8 Hz).

H. (3-{1-[5-(2-Fluoro-phenylethynyl)-furan-2-carbonyl]-piperidin-4-yl}-benzyl)-carbamic acid tert-butyl ester

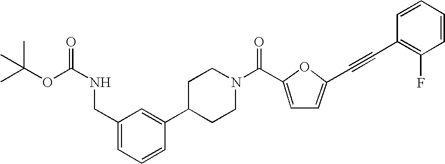

To a suspension of 2.75 g (11.96 mmol) of 5-(2-Fluoro-phenylethynyl)-furan-2-carboxylic acid in 100 mL of dichloromethane at 0° C. is added 1.25 mL (14.35 mmol) of oxalylchloride dropwise followed by 3-4 drops of dimethylformamide. The mixture is stirred for 15 minutes at 0° C. then allowed to warm to room temperature over 2 hours. After all solids are dissolved into solution the, mixture is concentrated in vacuo to afford the 5-(2-Fluoro-phenylethynyl)-furan-2-carbonyl chloride (100%).

This material is dissolved in 100 mL of dichlorornethane and treated with 3.7 g (12.74 mmol) of (3-Piperidin-4-yl-benzyl)-carbamic acid tert-butyl ester as a solution in 24 mL of dichloromethane dropwise, followed by 2.5 mL of triethylamine. The resulting mixture is stirred for 2 hours, then washed with 100 mL of water, followed by 100 mL 0.5 M HCl and 100 mL of saturated NaHCO₃. The organic phase is separated, dried (MgSO₄) and concentrated in vacuo to afford 7.11 g of a brown solid. The residue is flash chromatographed over 100 g SiO₂ (eluted with EtOAc:Hept=1:1) to afford 4.85 g (81%) of title compound as a white solid. ¹H NMR (300 MHz, CDCl₃) δ 7.53 (td, 1H), 7.41-7.28 (m, 2H), 7.18-7.08 (m, 5H), 7.04 (d, 1H, J=3.6 Hz), 6.76 (d, 1H, J=3.6 Hz), 4.83 (bs, 1H), 4.72 (bm, 2H), 4.31 (d, 2H, J=5.6 Hz), 3.1 (bs, 1H), 2.82 (m, 2H), 1.96 (m, 2H), 1.79 (td, 2H, J=12.8, 4.0 Hz), 1.46 (s, 9H).

I. [4-(3-Aminomethylphenyl)piperidin-1-yl]-[5-(2-fluorophenylethynyl)furan-2-yl]-methanone trifluoroacetate [which may also be named as 3-(1-[5-(2-fluorophenylethynyl)fuiran-2-carbonyl]-piperidine-4-yl)benzylamine trifluoroacetate]

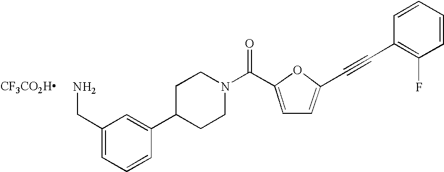

(3-{1-[5-(2-Fluorophenylethynyl)furan-2-carbonyl]-piperidin-4-yl}benzyl)carbamic acid tert-butyl ester (1.1 g, 2.19 mmol) is treated with 60 mL of 10% trifluoroacetic acid in dichloromethane and stirred overnight. The mixture is concentrated in vacuo to afford a quantitative yield of [4-(3-Aminomethylphenyl)-piperidin-1-yl]-[5-(2-fluorophenylethynyl)-furan-2-yl]-methanone trifluoroacetate. ¹H NMR (300 MHz, DMSO-d6) d 1.6 (m, 2H), 1.85 (br-d, 2H), 2.85 (m, 2H), 2.8-3.4 (br-s, 1H), 4.0 (dd, 2H), 4.4 (br-s, 2H), 7.1 (dd, 2H), 7.25-7.4 (m, 6H) 7.55 (m, 1H), 7.7 (t, 1H), 8.2 (br-s, 3H). LC-MS (ESI) m/z 403 (M⁺+1, 100). HPLC (BDS Hypersil C18, 50 mm×4.6 mm, particle size: 3 μm, flow: 1.0 mL/min, eluents: A: H₂O/0.05% TFA, B: CH₃CN/0.05% TFA, timetable: 0 min (95% A /5% B), 3 min, (10% A /90% B): Rt 2.54 min, purity 100% by area at 210 nm.

### Example 2

### [4-(3-Aminomethylphenyl)piperidin-1-yl]-[5-(2-fluorophenylethynyl)furan-2-yl]-methanone methanesulfonate [which may also be named as 3-(1-[5-(2-fluorophenylethynyl)fuiran-2-carbonyl]-piperidine-4-yl)benzylamine methanesulfonate]

A. 4-Oxo-piperidine-1-carboxylic acid 2-trimethylsilanylethyl ester

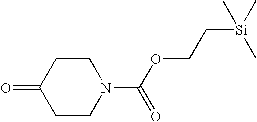

To a stirred suspension of 1,1′-carbonyldiimidazole (316.46 g, 1.95 mol) in dry MeCN (3.5 L) under N₂ is added 2-(trimethylsilyl)ethanol (240 mL, 1.67 mol) drop-wise over 45 minutes; the reaction temperature increased from 19.7 to 27.8° C. After 15 minutes 4-piperidone monohydrate hydrochloride (260.9 g, 1.70 mol) and Et₃N (250 mL, 1.79 mol) are added and the mixture is heated at reflux for 5.5 hours, the mixture is allowed to cool to room temperature overnight. The reaction mixture is partially concentrated in vacuo, diluted with H₂O and extracted 2 times with ether/EtOAc/-cyclohexane. The combined extracts are washed 2 times with 10% HCl, H₂O, saturated NaHCO₃ and H₂O. Concentration in vacuo and Kugelrohr distillation gives 298.5 g (73.4%) of title compound as a colorless liquid: bp 154-159° C. (0.9 mm); ¹H NMR (CDCl₃) δ 4.22 (m, 2H), 3.75 (t, 4H, J=6.2 Hz), 2.44 (t, 4H, J=6.2 Hz), 1.02 (m, 2H), 0.04 (s, 9H); GC/MS (8.6 min) m/z 200, 73 (100).

B. Trifluoromethanesulfonyloxy-3,6-dihydro-2H-pyridine-1-carboxylic acid 2-trimethylsilanylethyl ester

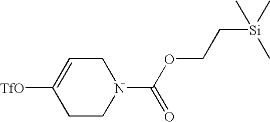

1 M Li(TMS)₂/THF (485 mL) is diluted with dry THF (1 L) under N₂ and the solution is cooled to −78° C. To this stirred solution is added drop-wise over 55 min a solution of 4-Oxo-piperidine-1-carboxylic acid 2-trimethylsilanyl-ethyl ester (106 g, 436 mmol) in dry THF (400 mL +75 mL rinse). After 45 minutes, a solution of N-phenylbis(trifluoromethanesulfonimide) (158.8 g, 445 mmol) in dry THF (700 mL+50 mL rinse) is added drop-wise over 45 minutes. After 1 hour at −78° C., the reaction mixture is placed in an ice-bath for 3 hours, then concentrated in vacuo. The residue is diluted with H₂O and the aqueous layer is extracted 2 times with ether/cyclohexane. The combined organic layers are dried (MgSO₄) and placed in the freezer overnight. Concentration in vacuo gives 158.6 g (97%) of title compound as an amber oil: ¹H NMR (CDCl₃) δ 5.78 (bs, 1H), 4.21 (m, 2H), 4.10 (m, 2H), 3.68 (t, 2H, J=5.3 Hz), 2.46 (m, 2H), 1.02 (m, 2H), 0.05 (s, 9H).

C. 4-(3-Cyanophenyl)-3,6-dihydro-2H-pyridine-1-carboxylic acid 2-trimethylsilanyl-ethyl ester

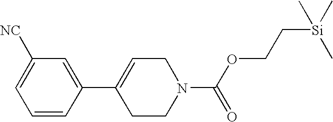

To a mechanically stirred mixture of trifluoromethanesulfonyloxy-3,6-dihydro-2H-pyridine-1-carboxylic acid 2-trimethylsilanyl-ethyl ester (158.6 g, 422 mmol) and 3-cyanophenylboronic acid (66.4 g, 452 mmol) in MeCN (2.65 L) is added 2 M Na₂CO₃ (622 mL) and LiCl (53.8 g, 1.27 mol); much of the Na₂CO₃ precipitates out of solution. The mixture is deoxygenated by bubbling N₂ gas through it for 15 minutes, then Pd(Ph₃P)₄ (7.79 g, 6.74 mmol, 1.6 mol %) is added and the mixture is heated at reflux under N₂ for 3.5 hours. After cooling to rt overmight, the amber-red solution is decanted and partially concentrated in vacuo. The residue is filtered through filter aid (MeCN rinse) to remove olive green flakes of catalyst and then partially concentrated in vacuo. The residual oil is partitioned between EtOAc/n-heptane and 1 M Na₂CO₃ (200 mL) and the organic layer is washed with H₂O. Concentration in vacuo gives 140 g (138.8 g theory) of red oil. Flash chromatography (4:1 n-heptane/EtOAc) gives 90.7 g of title compound as a light amber oil. Early and late fractions are combined and partially concentrated in vacuo; addition of cyclohexane gives a fine white precipitate, which is removed by filtration. Concentration in vacuo and flash chromatography (83:17 n-heptane/EtOAc) gives 16.17 g (77% total) of additional title compound as a light yellow oil. IR (KBr) νₘₐₓ 2952, 2229, 1699, 1433, 1249, 1235, 861, 839 cm⁻¹; ¹H NMR (CDCl₃) δ 7.65-7.52 (m, 3H), 7.44 (t, 1H, J=7.7 Hz), 6.11 (bs, 1H), 4.23 (m, 2H), 4.15 (m, 2H), 3.70 (t, 2H, J=5.6 Hz), 2.52 (m, 2 H), 1.04 (m, 2H), 0.06 (s, 9H); MS (ESI, MeOH/H₂O, infusion) m/z 347, 346 (M+NH₄)⁺, 328 (M⁺), 327 (M⁺−1, 100), 317, 315, 302, 301. Anal. Calcd for C₁₈H₂₄N₂O₂Si (328.46): C, 65.82; H, 7.36; N, 8.53. Found: C, 65.47; H, 7.43; N, 8.46.

D. 4-(3-Aminomethylphenyl)piperidine-1-carboxylic acid 2-trimethylsilanylethyl ester hydrochloride

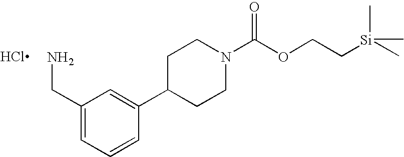

and 4-(3-Cyanophenyl)piperidine-1-carboxylic acid 2-trimethylsilanylethyl ester

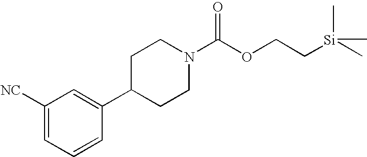

A mixture of 4-(3-Cyanophenyl)-3,6-dihydro-2H-pyridine-1-carboxylic acid 2-trimethylsilanylethyl ester (255.1 g, 776.6 mmol) and 10% Pd/C (25.1 g) in absolute EtOH (5 L) and concentrated HCl (80 mL, 960 mmol) is hydrogenated in an autoclave at 70-75 psi H₂ gas for 43 hours. The catalyst is removed by filtration (EtOH rinse) and the filtrate concentrated in vacuo to give a beige semi-solid, which is suspended in 3:1 ether/EtOAc and stirred for 1.5 hours, breaking up the clumps. Filtration gives 207.65 g of 4-(3-aminomethylphenyl)piperidine-1-carboxylic acid 2-trimethylsilanylethyl ester hydrochloride as an ivory solid. Concentration in vacuo of the filtrate results in the precipitation of a white solid from the residual oil; addition of ether and filtration gives additional product (4.63 g, 73.7% overall): mp 169-173° C.; IR (KBr) νₘₐₓ 2952, 1700, 1435, 1239, 1220 cm⁻¹; ¹H NMR (CD₃OD) δ 7.41-7.27 (m, 4H), 4.26 (dm, 2H, J=13.5 Hz), 4.20 (m, 2H), 4.09 (s, 2H), 2.92 (bm, 2H), 2.79 (tt, 1H, J=12.1, 3.6 Hz), 1.84 (dm, 2H, J=12.9 Hz), 1.62 (qd, 2H, J=12.6, 4.1 Hz), 1.02 (m, 2H), 0.06 (s, 9H); MS (APCI, MeOH/H₂O) m/z 336, 335 (M⁺+1, 100), 191. Anal. Calcd for C₁₈H₃₀N₂O₂Si.HCl (370.96): C, 58.28; H, 8.42; N, 7.55. Found: C, 58.20; H, 8.28; N, 7.59]. Concentration of the filtrate gives 55 g (21.4%) of 4-(3-Cyanophenyl)piperidine-1-carboxylic acid 2-trimethylsilanylethyl ester as an amber oil. Alternatively, for some runs, the procedure described herein may yield 4-(3-Aminomethyl-phenyl)-piperidine-1-carboxylic acid 2-trimethylsilanyl-ethyl ester hydrochloride.

E. 4-[3-(tert-Butoxycarbonylaminomethyl)phenyl]piperidine-1-carboxylic acid 2-trimethylsilanylethyl ester

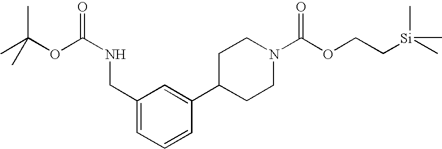

Method 1(aminomethyl derivative as the starting material): A solution of 4-(3-Aminomethyl-phenyl)piperidine-1-carboxylic acid 2-trimethylsilanylethyl ester hydrochloride in H₂O (1 L) is treated with NaHCO₃ (67.38 g, 802 mmol) and THF (1.3 L); a solution of Boc₂O (153 mL, 666 mmol) in THF (200 mL, +200 mL rinse) is added in 3 portions. The mixture is allowed to stir at room temperature for 1.75 hours, then partially concentrated in vacuo. The residue is diluted with H₂O and the aqueous layer is extracted 2 times with ether/cyclohexane. The combined extracts are washed with H₂O, brine, and dried (MgSO₄). Concentration in vacuo and addition of n-pentane and seeding with pure 4-[3-(tert-Butoxycarbonylaminomethyl)phenyl]piperidine-1-carboxylic acid 2-trimethylsilanylethyl ester gives 267 g of a tacky solid. The solid is covered with ice-cold n-pentane and the solid is crushed. The mixture is filtered and washed with ice-cold n-pentane to give 231 g (93%) of title compound as a colorless solid. Recrystallization from hexane/cyclohexane gives white crystals: mp 76-78.5° C; IR (KBr) νₘₐₓ 2951, 1698, 1249, 1219, 1173 cm⁻¹; ¹H NMR (CDCl₃) δ 7.26 (m, 1H), 7.10 (m, 3H), 4.85 (bs, 1H), 4.29 (d, 4H, J=5.8 Hz), 4.19 (m, 2H), 2.83 (t, 2H, J=12.5 Hz), 2.64 (tt, 1H, J=12.0, 3.6 Hz), 1.81 (m, 2H), 1.60 (m, 2H), 1.45 (s, 9H), 1.01 (t, 2H, J=8.4 Hz), 0.04 (s, 9H); MS (APCI, MeOH/H₂O) m/z 433 (M−1)⁻, 378, 377 [(M−1-C₄H₈)⁻, 100], 333, 259; HPLC, 21.8 min. Anal. Calcd for C₂₃H₃₈N₂O₄Si (434.65): C, 63.56; H, 8.81; N, 6.45; Found: C, 63.58; H, 8.59; N, 6.36.

Method 2 (cyanophenyl derivative as the starting material): To a stirred solution of 4-(3-Cyanophenyl)-piperidine-1-carboxylic acid 2-trimethylsilanylethyl ester (115.85 g, 350 mmol) and NiCl₂.6H₂O (50.8 g, 213 mmol) in MeOH (1.7 L) at 0° C. is added a suspension/solution ofanhydrous NiCl₂ (17.9 g, 138 mmol) and H₂O (15 mL, 832 mmol, 6 equivalents) in MeOH (30 mL) [there was not enough NiCl₂.6H₂O on hand]. Boc₂O (157.5 mL, 686 mmol) is added, then NaBH₄ (92.4 g, 2.44 mol) is added in portions over 1 hour. Caution: vigorous gas evolution! The black mixture is allowed to stir at room temperature overnight, then partially concentrated in vacuo. The residue is diluted with aqueous NaHCO₃ and EtOAc and filtered through filter aid (EtOAc rinse). The organic layer is washed with H₂O, brine and dried (MgSO₄). Concentration in vacuo gives 159 g of opaque oil, which is dissolved in n-pentane (250 mL), seeded with pure 4-[3-(tert-Butoxycarbonylamino-methyl)-phenyl]piperidine-1-carboxylic acid 2-trimethylsilanyl-ethyl ester and placed in the freezer overnight. The mixture is filtered and washed with ice-cold n-pentane to give 37.5 g (24.6%) of the title compound as a colorless solid. Flash chromatography (83:17 n-heptane/EtOAc) gives additional product as white crystals after trituration with ice-cold n-pentane.

F. (3-Piperidin-4-yl-benzyl)carbamic acid tert-butyl ester

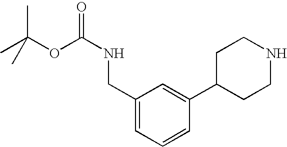

To a stirred solution of 4-[3-(tert-Butoxycarbonylaminomethyl)phenyl]piperidine-1-carboxylic acid 2-trimethylsilanylethyl ester (502 g, 1.155 mol) in THF (5 L) is added 1 M (n-Bu)₄NF/THF (1.44 L, 1.44 mol). The solution is warmed to 55° C. over 1 hour progressively (Caution: gas evolution!), then it is stirred at this temperature for additional 3 hours and is concentrated in vacuo to 1.05 kg of an amber oil. The concentrate is partitioned between H₂O (2 L) and Et₂O (1.5 L), and the aqueous phase is acidified to pH 4 by the addition of 2 N HCl. The organic phase is eliminated. The aqueous phase is basified with 50% aq. NaOH solution to pH 13-14 and is extracted with two portions of Et₂O (once with 2.0 L, and once with 1.0 L). The organic phase is washed with brine (0.5 L), dried (MgSO₄) and concentrated to 373 g of an amber oil. To this crude material is added n-pentane (250 mL), then with stirring 250 mL of Et₂O is added progressively, and the mixture is seeded with 50 mg of previously prepared (3-Piperidin-4-yl-benzyl)carbamic acid tert-butyl ester. The resulting thick suspension is allowed to stand over-night at room temperature, then the solid is isolated by filtration, washed with 250 mL of a n-pentane/Et₂O (1:1) mixture, and dried at 30° C. under 50 mm Hg for 24 hours, to afford 315.6 g of the title compound (81%), as an off-white solid: mp 79-84° C.; ¹H NMR (300 MHz, CDCl₃) δ 7.25 (m, 1H), 7.07-7.13 (m, 3H), 4.85 (bs, 1H), 4.29 (d, 2H, J=5.1 Hz), 3.17 (dm, 2H, J=12.0 Hz), 2.72 (td, 2H, J=12.0, 2.4 Hz), 2.60 (tt, 1H,J=12.0, 3.6 Hz), 1.81 (m, 2H), 1.55-1.70 (m, 3H), 1.46 (s, 9H); MS (APCI, MeOH/H₂O) m/z 292, 291 (M⁺+1, 100). Anal. Calcd for C₁₇H₂₆N₂O₂: C, 70.31; H, 9.02; N, 9.65. Found: C, 69.74; H, 9.47; N, 9.42.

G. 5-(2-Fluorophenylethynyl)-furan-2-carboxylic acid methyl ester

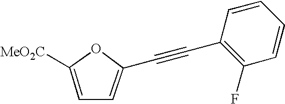

A 3-L, 3-necked round-bottomed flask equipped with a mechanical stirrer, reflux condenser and a temperature probe is charged with methyl 5-bromo-2-furoate, (146.76 g, 0.716 mol), 1-ethynyl-2-fluorobenzene (86 g, 0.716 mol), THF (1.03 L) and copper (I) iodide (1.36 g, 7.16 mmol, 0.01 equiv.). The system is flushed with N₂ and stirred. Dichlorobis(triphenylphosphine) palladium (II) (5.03 g, 7.16 mmol, 0.01 equiv.) is then added, followed by triethylamine (0.504 L, 3.58 mol, 5 equiv.) and the system is purged (vacuum/N₂) 3 times. The reaction mixture is heated to 45° C., then the heat source is turned off, and the exotherm is allowed to warm the mixture to 65° C. (mild reflux). After 30 minutes the exotherm ceases, the heat source is turned on, and the reaction mixture is maintained at 65° C. for an additional 1.5 hours. The reaction mixture is allowed to cool to 60° C., 20 mL of MeOH and 82 g of charcoal (DARCO G-60) are added, and the mixture is stirred at 60-65° C. for 45 minutes (some gas evolution observed). The mixture is cooled to 40° C. and the solids are eliminated by filtration after rinsing with 0.3 L of EtOAc. The filtrate is concentrated on rotary evaporator to 187 g of a yellow solid. The crude material is recrystallized from 1.2 L of i-PrOH. The filter cake is washed with 0.3 L of i-PrOH, and dried (50° C./50 mm Hg, N₂ bleed, 4 hours) to afford 127.1 g (73%) of 5-(2-fluorophenylethynyl)-furan-2-carboxylic acid methyl ester as a light beige solid: mp 106-108° C.; ¹H NMR (300 MHz, DMSO-d₆) δ 7.66 (m, 1H), 7.53 (m, 1H), 7.38 (d, 1H, J=3.7 Hz), 7.35 (m, 1H), 7.28 (t, 1H, J=7.6 Hz), 7.11 (d, 1H, J=3.7 Hz), 3.83 (s, 3H); LC-MS (ESI) m/z 245 (M⁺+1, 100). Anal. Calcd for C₁₄H₉FO₃: C, 68.85; H, 3.71. Found: C, 68.69; H, 3.75.

H. 5-(2-Fluorophenylethynyl)-furan-2-carboxylic acid

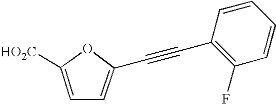

To a stirred solution of 5-(2-fluorophenylethynyl)-furan-2-carboxylic acid methyl ester (307.8 g, 1.26 mol) in a THF (3.0 L)/MeOH (0.75 L) mixture, at 8° C., is added 0.755 L of a 2.5N aqueous NaOH solution (1.88 mol, 1.5 equiv.). The exotherm is allowed to warm the reaction mixture to 19° C., and the mixture is stirred for additional 2 hours at 20° C. (±2° C.). The mixture is partitioned between EtOAc (4.0 L) and H₂O (4 L) and the phases are separated. The aqueous phase is acidified with 6N aqueous HCl solution to pH 1, and extracted with 4 L of EtOAc. The organic phase is washed with brine (1 L), dried (MgSO₄), and concentrated on a rotary evaporator (bath at 50° C.) till solid precipitated (volume approx. 1 L). The solid is kept at 4° C. over-night, then the solid is isolated by filtration, rinsed with 0.3 L of cold EtOAc, and dried at room temperature under 50 mm Hg for 70 hours to afford 235.0 g of 5-(2-fluorophenylethynyl)-furan-2-carboxylic acid as a white solid: mp 196-198° C.; ¹H NMR (300 MHz, DMSO-d₆) δ 13.4 (bs, 1H), 7.69 (m, 1H), 7.55 (m, 1H), 7.38 (t, 1H, J=9.0 Hz), 7.32 (d, 1H, J=3.8 Hz), 7.31 (t, 1H, J=7.5 Hz), 7.11 (d, 1H, J=3.8 Hz); LC-MS (H₂O/CH₃CN, ESI) m/z 272 (M⁺+1+CH₃CN, 100), 231 (M⁺+1, 15). Anal. Calcd for C₁₃H₇FO₃: C, 67.83; H, 3.07. Found: C, 67.55; H, 2.95.

The mother liquor is concentrated on rotary evaporator (bath at 50° C.) to approx. ½ volume and cooled to room temperature. The second crop of 23.1 g of product is isolated after filtration, rinsing the cake with 100 mL of EtOAc, and drying for 2 hours at 50° C. /50 mm Hg. Combined yield for 2 crops is 258.1 g, 89%.

I. 5-(2-Fluorophenylethynyl)-furan-2-carbonyl chloride

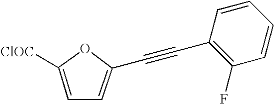

To a suspension of the 5-(2-fluorophenylethynyl)furan-2-carboxylic acid (233.8 g, 1.015 mol) in methylene chloride (2.35 L) containing 2.5 g of DMF, under N₂, oxalyl chloride (176.4 g, 1.390 mol, 1.37 eq.) is added dropwise over 45 minutes at 22° C. (±2° C.). The mixture is stirred for additional 2.45 hours at room temperature (rt), then it is concentrated under in vacuo. The crude product is dried overnight (rt/50 mm Hg) to 256.0 g of 5-(2-fluorophenylethynyl)furan-2-carbonyl chloride (101%, includes DMF) as a light cream color solid: mp 96-98° C.; ¹H NMR (300 MHz, CDCl₃) δ 7.55 (td, 1H, J=7.3, 1.8 Hz), 7.50 (d, 1H, J=3.8 Hz), 7.46-7.38 (m, 1H), 7.21-7.11 (m, 2H), 6.83 (d, 1H, J=3.8 Hz. Anal. Calcd for C₁₃H₆ClFO₂: C, 62.80; H, 2.43. Found: C, 62.48; H, 2.44.

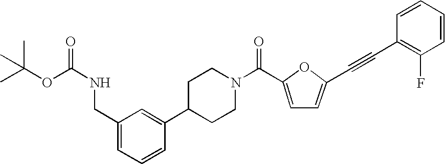

J. (3-{1-[5-(2-Fluoro-phenylethynyl)-furan-2-carbonyl]-piperidin-4-yl}-benzyl)-carbamic acid tert-butyl ester

To a solution of (3-Piperidin-4-yl-benzyl)-carbamic acid tert-butyl ester (151.1 g, 0.52 mol) and triethylamine (93 mL, 0.66 mol) in methylene chloride (2 L), under N₂, is added dropwise, over 1 hour, at 10° C. (±2° C.), a solution of 5-(2-fluorophenylethynyl)-furan-2-carbonyl chloride in methylene chloride (0.6 L). Following the end of addition the ice-bath is removed and the solution is stirred at room temperature for 3 hours. The solution is washed with water (0.9 L), 0.25 N aq. HCl (0.9 L), saturated aq. NaHCO₃ (0.5 L), brine (0.5 L), dried (MgSO₄), and concentrated in vacuo to 291.6 g of a foaming oil. The residue is crystallized from methylene chloride (0.5 L)/heptane (1.25 L) mixture. The solid is isolated by filtration, rinsed with 0.9 L of a heptane/methylene chloride (2:1) mixture, and dried at 35° C. under 50 mmHg for 16 hours to give 162.6 g of the title compound as a white solid: mp 111-113° C.; ¹H NMR (300 MHz, CDCl₃) δ 7.53 (td, 1H), 7.41-7.28 (m, 2H), 7.18-7.08 (m, 5H), 7.04 (d, 1H, J=3.6 Hz), 6.76 (d, 1H, J=3.6 Hz), 4.83 (bs, 1H), 4.72 (bm, 2H), 4.31 (d, 2H, J=5.6 Hz), 3.1 (bs, 1H), 2.82 (m, 2H), 1.96 (m, 2H), 1.79 (td, 2H, J=12.8, 4.0 Hz), 1.46 (s, 9H); LC-MS (ESI) m/z 503 (M⁺+1, 100). Anal. Calcd for C₃₀H₃₁N₂O₄F (502.59): C, 71.70; H, 6.22; N, 5.57. Found: C, 71.66; H, 6.51; N, 5.52.

The mother liquor is partially concentrated removing the bulk of methylene chloride. The resulting solid suspension is isolated by filtration, rinsed with heptane and dried (35° C., 50 mm Hg, 40 hours), to afford an additional 86.5 g of the title compound as an off-white solid: mp 107-110° C. ¹H NMR and LC-MS data as above. Anal. Found: C, 71.39; H, 6.48; N, 5.37. Combined yield for 2 crops: 249.1 g, 96%.

K. [4-(3-Aminomethylphenyl)piperidin-1-yl]-[5-(2-fluorophenylethynyl)furan-2-yl]methanone methanesulfonate [which may also be named as 3-(1-[5-(2-fluorophenylethynyl)-furan-2-carbonyl]-piperidine-4-yl)benzylamine methanesulfonate]

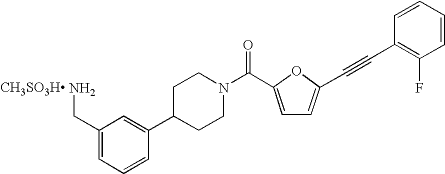

To a stirred suspension of (3-{1-[5-(2-Fluorophenylethynyl)furan-2-carbonyl]piperidin-4-yl}benzyl)-carbamic acid tert-butyl ester (459 g, 0.906 mol) in i-PrOH (2.25 L) at 40° C., is added methanesulfonic acid (60 mL, 88.8 g, 0.924 mol, 1.02 equiv.). The reaction mixture is heated progressively (Caution: CO₂ evolution!) over approx. 1 hour to 70° C. (suspension dissolves at 65-70° C., CO₂ evolution rate slows down). The mixture is stirred for 2 hours between 70 and 75° C. (suspension forms again), then it is allowed to cool to 35° C., and the solid is isolated by filtration. The filter cake is rinsed with 1 L of i-PrOH, then with 1 L of acetone, and dried (30° C./50 mm Hg/N₂ bleed, 70 hours) to afford 403.6 g (89.4%) of [4-(3-Aminomethyl-phenyl)piperidin-1-yl]-[5-(2-fluorophenylethynyl)furan-2-yl]methanone methanesulfonate, as a white crystalline solid: mp 172-174° C. ¹H NMR (300 MHz, DMSO-d₆) δ 8.12 (bs, 3H), 7.67 (tm, 1H, J=7.5 Hz), 7.54 (qm, 1H, J=7.0 Hz), 7.42-7.26(m, 6H), 7.10 (m, 2H), 4.34 (bs, 2H), 4.02 (q, 2H, J=5.5 Hz), 3.5-2.7 (bs, 2H), 2.89 (mn, 1H), 2.34 (s, 3H), 1.87 (m, 2H), 1.65 (m, 2H); ¹³C NMR (75 MHz, DMSO-d₆) δ 163.23, 159.92, 157.34, 147.99, 145.63, 135.84, 133.94, 133.21, 132.00, 131.89, 128.52, 127.03, 126.91, 126.59, 124.88, 117.55, 115.91, 115.64, 109.13, 108.93, 87.49, 83.47, 42.37, 41.64. LC-MS (ESI) m/z 404, 403 (M⁺+1, 100). Anal. Calcd for C₂₅H₂₃N₂O₂F.CH₃SO₃H: C, 62.64; H, 5.46; N, 5.62; F, 3.81; S, 6.43. Found: C, 62.64; H, 5.36; N, 5.63; F, 3.75; S, 6.48. Trace elements: Pd 2 ppm; P<2 ppm; Cu<1 ppm. HPLC (BDS Hypersil C18, 150mm×2mm, particle size: 3 μm, flow: 0.25 mL/min, eluents: A: H₂O/0.1% TFA, B: CH₃CN/0.1% TFA, timetable: 0 min (70% A/30% B), 15 min, (10% A/90% B): Rt 7.53 min, purity 98.3% by area at 225 nm.

### In vitro Test Procedure

As all the actions of tryptase, as described in the background section, are dependent on its catalytic activity, then compounds that inhibit its catalytic activity will potentially inhibit the actions of tryptase. Inhibition of this catalytic activity may be measured by the in vitro enzyme assay and the cellular assay.

Tr0yptase inhibition activity is confirmed using either isolated human lung tryptase or recombinant human β tryptase expressed in yeast cells. Essentially equivalent results are obtained using isolated native enzyme or the expressed enzyme. The assay procedure employs a 96 well microplate (Costar 3590) using L-pyroglutamyl-L-prolyl-L-arginine-para-nitroanilide (S2366: Quadratech) as substrate (essentially as described by McEuen et. al. Biochem Pharm, 1996, 52, pages 331-340). Assays are performed at room temperature using 0.5 mM substrate (2×Kₘ) and the microplate is read on a microplate reader (Beckman Biomek Plate reader) at 405 nm wavelength. The inhibition constants (Kᵢ) for the compound of the present invention and other structurally related compounds, are set forth in Table 1.

The unexpected superior properties of the compound of the present invention may be demonstrated by: 1) its superior β-Tryptase Inhibitory Potency (Kᵢ), and 2) its superior activity as measured in the Guinea Pig Model of Airway Hyperreactivity (Intravenous ID₅₀ and Oral ID₅₀).

Materials and Methods for Trtase Primary Screen (Chromogenic Assay)

Assay buffer

50 mM Tris (pH 8.2), 100 mM NaCl, 0.05% Tween 20, 50 μg/mL heparin.

Substrate

S2366 (Stock solutions of 2.5 mM).

Enzyme

Purified recombinant beta Tryptase Stocks of 310 μg/mL.

Protocol (Single Point Determination)

Add 60 μL of diluted substrate (final concentration of 500 μM in assay buffer) to each well

Add compound in duplicates, final concentration of 20 μM, volume 20 μL

Add enzyme at a final concentration of 50 ng/mL in a volume of 20 μL

Total volume for each well is 100 μL

Agitate briefly to mix and incubate at room temp in the dark for 30 minutes

Read absorbencies at 405 nM

Each plate has the following controls:

| | |
|---|---|
| Totals: | 60 μL of substrate, 20 μL of buffer (with 0.2% final |
| | concentration of DMSO), 20 μL of enzyme |
| Non-specific: | 60 μL of substrate, 40 μL of buffer (with 0.2% DMSO) |
| Totals: | 60 μL of substrate, 20 μL of buffer (No DMSO), |
| | 20 μL of enzyme |
| Non-specific: | 60 μL of substrate, 40 μL of buffer (No DMSO) |

Protocol (IC₅₀ and Kᵢ Determination)

The protocol is essentially the same as above except that the compound is added in duplicates at the following final concentrations: 0.01, 0.03, 0.1, 0.3, 1, 3, 10 uM (All dilutions carried out manually). For every assay, whether single point or IC₅₀ determination, a standard compound is used to derive IC₅₀ for comparison. From the IC₅₀ value, the Kᵢ can be calculated using the following formula:

Kᵢ=IC₅₀/(1+[Substrate]/Kₘ).

The greatest β-Tryptase inhibitory potency disclosed in U.S. application Ser. No. 09/843,126 is that for [4-(3-Aminomethylphenyl)piperidin-1-yl]-(3,4-dichlorophenyl) methanonetrifluoroacetate which has a Tryptase Kᵢ of 31 nM. The compound of formula (I), [also denoted as the compound of Structure (III) where X=F], shows a range of potency between 7.6 nM and 9.7 nM. This represents a 3.2 to 4.08 fold increase in β-Tryptase inhibitory potency from the most potent compound disclosed in U.S. application Ser. No. 09/843,126.

Additionally, Kᵢ values with respect to tryptase for the compound of the present invention, and three structurally related comparison compounds are set forth in Table 1 below:

### Comparison Between Phenylethynyl Compounds and Flourinated Phenyethynyl Compounds

The compound of Structure (II) where X=H, which has a phenyl ring in place of the furan ring of the compound of the present invention and does not have a fluoro group on the phenylethynyl moiety, has a β-tryptase Kᵢ of 106 nM; and the compound of Structure (II) where X=F, which has a phenyl ring in place of the furan ring of the compound of the present invention and contains an ortho-fluoro group on the phenylethynyl moiety, has a β-tryptase Kᵢ of 97 nM.

Thus, the data demonstrate that the fluorination of the phenylethynyl moiety only has a marginal effect because there is only a 1.09-fold increase in β-Tryptase inhibitory potency between the Compound of Structure (II) where X=H and the Compound of Structure (II) where X=F.

However, the Compound of Structure (III) where X=H, which is the des-fluoro analog of the compound of the present invention, has a β-tryptase Kᵢ of 31 nM; and the Compound of formula (I) of the present invention, containing an ortho-fluoro group on the phenylethynyl moiety, has a β-tryptase Kᵢ of 7.6 nM and 9.7 nM.

Thus, the data demonstrate unexpectedly superior results. The fluorination of the phenylethynyl moiety represents a 3.20 to 4.08-fold increase in β-Tryptase inhibitory potency between the Compound of Structure (III) where X=H and the Compound of formula (I) of the present invention.

### Comparison Between Phenyl Compounds and Furan Compounds

The comparison of data provided in Table 1 between the Compound of Structure (II) where X=H β-tryptase Kᵢ of 106 nM) and the Compound of Structure (III) where X=H (β-tryptase Kᵢ of 31 nM) demonstrates only a 3.42-fold increase in β-Tryptase inhibitory potency.

However, the comparison of data provided in Table 1 between the Compound of Sructure (II) where X=F (β-tryptase Kᵢ of 97 nM) and the Compound of formula (I) of the present invention (β-tryptase Kᵢ of 7.6 nM and 9.7 nM) demonstrate a significant 10 to 12.7-fold increase in β-Tryptase inhibitory potency. Thus, the data demonstrate unexpectedly superior results.

TABLE 1
β-Tryptase Inhibition (chromogenic assay)

| | β-tryptase Kᵢ (nM) | |
|---|---|---|
| | * = Denotes the compound of the present | |
| | invention. | |
| Structure | X = H | X = F |
| 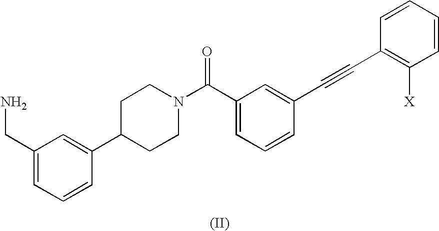 | 106 [4-(3-aminomethyl- phenyl)piperidin-1- yl]-(3-phenylethynyl- phenyl)methanone trifluoroacetate | 97 [4-(3-aminomethyl- phenyl)piperidin-1- yl]-[3-(2- fluorophenylethynyl)- phenyl]methanone trifluoroacetate |
| 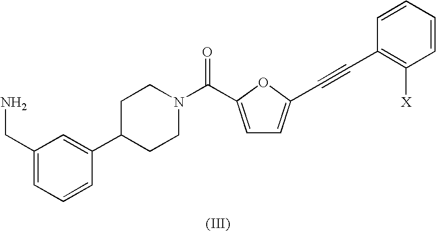 | 31 [4-(3-aminomethyl- phenyl)piperidin-1- yl]-(5-phenylethynyl- furan-2-yl)- methanone trifluoroacetate | 7.6, 9.7 [4-(3-aminomethyl- phenyl)piperidin-1- yl]-[5-(2- fluorophenyl- ethynyl)furan-2-yl]- methanone trifluoroacetate* |

Additional experiments using the Guinea Pig Model of Airway Hyperreactivity (Intravenous ID₅₀ and Oral ID₅₀) for the compound of the present invention, and the most closely related β-Tryptase compounds disclosed in U.S. application Ser. No. 09/843,126 reveal unexpectedly superior properties for the compound of the present invention.

### In vivo Test Procedure

Assay Protocol:

Sensitization and drug treatment: Male Hartley guinea pigs (225-250g) are sensitized with ovalbumin (0.5 mL of 1% solution, i.p. and s.c.). On day 4, animals received a booster injection (i.p.) of 0.5 mL of 1% ovalbumin. On day 21, animals are orally dosed (2 ml/kg) with either vehicle (0.5% methylcellulose/0.2% Tween 80) or test compound 2 hours prior to antigen challenge. Thirty minutes before antigen challenge the animals are also injected with mepyramine (30 mg/kg, i.p.) to prevent anaphylactic collapse. Animals are then exposed for 5 minutes to an aerosol of either saline (control animals) or 1% ovalbumin using a deVilbiss Ultraneb nebulizer.

AHR measurement: Eighteen to twenty four hours after challenge, animals are anesthetized with a combination of ketamine (133 mg/kg) and xylazine (24 mg/kg) given intramuscularly, surgically prepared and then mounted in a whole body plethysmograph for lung function measurement. Animals were connected to Ugo-Basile ventilators delivering a tidal volume of 1 mL/100 g at a rate of 50 breaths/minute via a tracheal cannula. The jugular vein is also cannulated for histamine challenge. A water filled oesophageal cannula is placed such that transpulmonary pressure could be recorded. Transpulmonary pressure is measured as the difference between the tracheal and esophageal cannulas using a differential pressure transducer. The volume, airflow, and transpulmonary pressure signals are monitored using a pulmonary analysis system (Buxco XA software) and used to calculate pulmonary resistance (cm H₂O/mL/s) and dynamic compliance (mL/cm H₂O). Airway resistance and dynamic compliance were computed on a breath by breath basis. Histamine is administered intravenously and reactivity to increasing concentrations (0.1-10 μg/kg) assessed. ID₅₀'s are estimated from the R_{L300} (histamine concentration required to 300% increase in lung resistance) and C_{dyn40} (histamine concentration required to induce a 40% decrease in dynamic lung compliance) parameters deduced from the individual induce a histamine dose-response curves.

Intravenous and oral data in Table 2 show the unexpectedly superior properties for performance of the compound of the present invention [also denoted as the compound of Structure (III) where X=F] in the guinea pig model of airway hyperresponsiveness.

TABLE 2
Guinea Pig Model of Airway Hyperreactivity

| | Intravenous ID₅₀ (mg/kg) | | Oral ID₅₀ (mg/kg) | |
|---|---|---|---|---|
| Compound Name | | Dynamic | | Dynamic |
| * = denotes the compound of the | Airway Hyper- | Lung | Airway Hyper- | Lung |
| present invention | responsiveness | Compliance | responsiveness | Compliance |
| [4-(3-aminomethylphenyl)- | >3.0 | >3.0 | ND | ND |
| piperidin-1-yl]-(3- | | | | |
| phenylethynylphenyl)- | | | | |
| methanone trifluroacetate | | | | |
| Structure (II) where X = H | | | | |
| [4-(3-aminomethylphenyl)- | 1-3 | >3.0 | Protective at 3, | >30 |
| piperidin-1-yl]-(5- | | | 10 & 30 but not | |
| phenylethynylfuran-2-yl)- | | | statistically | |
| methanone trifluroacetate | | | significant | |
| Structure (III) where X = H | | | | |
| [4-(3-Aminomethylphenyl)- | <0.3 | <0.3 | 1-2 | 1-2 |
| piperidin-1-yl]-[5-(2-fluoro- | | | | |
| phenylethynyl)furan-2-yl]- | | | | |
| methanone trifluroacetate* | | | | |
| Structure (III) where X = F | | | | |

Efficacy of the compound of the present invention [Structure (III) where X=F], is profiled on Airway Hyperresponsiveness (AHR) to histamine in sensitized guinea pigs, via both intravenous and oral routes. The compound of the present invention has no effect on basal airway resistance or basal dynamic lung compliance. Sensitization and challenge with ovalbumin results in an increase in bronchial reactivity to histamine as denoted by a significant decrease in R_{L300} or PC₃₀₀ (provocative challenge required to elicit a 300% increase in airway resistance or R_{L}). Upon intravenous dosing at 0.3, 1 and 3 mg/kg, the compound of the present invention is protective at all doses as measured by airway resistance and lung compliance (ID₅₀<0.3 mg/kg).

In contrast the less potent β-trypase inhibitor compounds of Structure (II) where X=H and Structure (III) where X=H does not show any protective effects as measured by lung compliance. The latter reversed airway resistance at the highest dose with an ID₅₀ of 1-3 mg/kg, while the former is uniformly ineffective (Table 2).

Upon oral dosing, 2 hours prior to ovalbumin challenge, the compound of the present invention significantly protects against AHR to histamine with an ID₅₀ =1-2 mg/kg as measured by airway resistance and dynamic lung compliance. In contrast the compound of Structure (III) where X=H, dosed orally at 3, 10, and 30 mg/kg is protective on airway resistance, but the effects are not statistically significant. No effect on lung compliance is observed.

Taken together, the intravenous and oral data of the compound of the present invention in the guinea pigmodel of airway hyperresponsiveness clearly show that the compound of the present invention exhibits unexpectedly superior tryptase inhibition activity over compounds in U.S. application Ser. No. 09/843,126. Consequently, the compound of the present invention readily has applications in pharmaceutical compositions for treating a wide variety of tryptase related conditions, and naturally, in methods for treating such conditions in patients.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A compound of Formula (I): 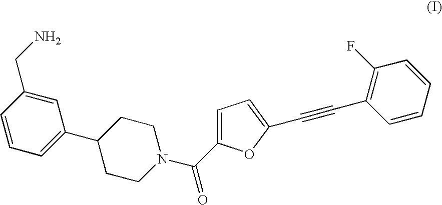
or a prodrug, pharmaceutically acceptable salt, or solvate thereof.

2. The compound of claim 1 as a pharmaceutically acceptable salt thereof.

3. The compound of claim 2 wherein the pharmaceutically acceptable salt is a trifluoroacetate or methanesulfonate salt.

4. A method for treating a patient suffering from, or subject to, a physiological condition in need of amelioration comprising administering to the patient a therapeutically effective amount of the compound of claim 1.

5. The method of claim 4, wherein the physiological condition is selected from the group consisting of inflammatory disease, a disease of joint cartilage destruction, ocular conjunctivitis, vernal conjunctivitis, inflammatory bowel disease, asthma, allergic rhinitis, interstitial lung disease, fibrosis, sceleroderma, pulmonary fibrosis, liver cirrhosis, myocardial fibrosis, neurofibroma, hypertrophic scar, dermatological condition, condition related to atherosclerotic plaque rupture, periodontal disease, diabetic retinopathy, tumor growth, anaphylaxis, multiple sclerosis, peptic ulcer, and syncytial viral infection.

6. The method of claim 5, wherein the physiological condition is inflammatory disease.

7. The method of claim 6 wherein the inflammatory disease is joint inflammation, arthritis, rheumatoid arthritis, rheumatoid spondylitis, gouty arthritis, traumatic arthritis, rubella arthritis, psoriatic arthritis, or osteoarthritis.

8. The method of claim 5, wherein the physiological condition is a dermatological condition.

9. The method of claim 8, wherein the dermatological condition is atopic dermatitis or psoriasis.

10. The method of claim 5, wherein the physiological condition is related to atherosclerotic plaque rupture.

11. The method of claim 10, wherein the physiological condition related to atherosclerotic plaque rupture is myocardial infarction, stroke, or angina.

12. A method for treating a patient suffering from asthma, comprising administering to the patient a combination of a thereapeutically effective amount of a compound of claim 1, and a second compound selected from the group consisting of a beta andrenergic agonist, anticholinergic, anti-inflammatory corticosteroid, and anti-inflammatory agent.

13. A pharmaceutical composition comprising a therapeutically effective amount of a compound of claim 1 and a pharmaceutically acceptable carrier thereof.

14. A pharmaceutical composition comprising a compound of claim 1 and a therapeutically effective amount of a second compound selected from the group consisting of a beta andrenergic agonist, anticholinergic, anti-inflammatory corticosteroid, and anti-inflammatory agent; and a pharmaceutically acceptable carrier.

15. The pharmaceutical composition of claim 14, wherein the second compound is a beta andrenergic agonist.

16. The pharmaceutical composition of claim 15, wherein the beta andrenergic agonist is selected from albuterol, terbutaline, formoterol, fenoterol, or prenaline.

17. The pharmaceutical composition of claim 14, wherein the second compound is an anticholinergic.

18. The pharmaceutical composition of claim 17, wherein the anticholinergic is ipratropium bromide.

19. The pharmaceutical composition of claim 14, wherein the second compound is an anti-inflammatory corticosteroid.

20. The pharmaceutical composition of claim 19, wherein the anti-inflammatory corticosteroid is selected from beclomethasone dipropionate, triamcinolone acetonide, flunisolide or dexamethasone.

21. The pharmaceutical composition of claim 14, wherein the second compound is an anti-inflammatory agent.

22. The pharmaceutical composition of claim 21, wherein the anti-inflammatory agent is soldium cromoglycate or nedocromil sodium.

23. The pharmaceutical composition of claim 14, wherein the second compound is a pharmaceutically acceptable carrier thereof.
